(19) United States
(12) Patent Application Publication
(10) Pub. No.: US 2003/0059489 A1
(43) Pub. Date: Mar. 27, 2003

(54) Genital lubricating compositions and uses thereof

(75) Inventors: Lucie Elise Letourneau, Ville St-Laurent (CA); Shirley Duplessis, Ville St-Laurent (CA)

Correspondence Address: PEPPER, HAMILTON LLP, 600 Fourteenth Street, N.W., Washington, DC, 20005-2004, US

(73) Assignee: CANOLIO INC.

(21) Appl. No.: 09/961,100

(22) Filed: Sep. 24, 2001

(51) Int. Cl. A61K 35/78; A61K 31/225

(52) U.S. Cl. 424/776; 514/547

(57) ABSTRACT

This invention relates to a genital lubricant composition and to the uses thereof. The composition comprises i) fatty acids and/or homeopathic dilutions of plant extracts and ii) a physiologically acceptable carrier. According to a preferred embodiment, the genital lubricant composition comprises about 0.05% to about 0.5% hemp seed oil and a physiologically acceptable carrier so that it forms a lotion, a cream or a gel. The composition according to the invention is particularly useful for use as a vaginal moisturizer, or as a personal lubricant for use prior or during sexual intercourse.

### A) FIELD OF THE INVENTION

The present invention relates to a genital lubricant composition that is particularly useful when used in vaginal moisturizers and in personal lubricants.

### B) BRIEF DESCRIPTION OF THE PRIOR ART

There is presently on the market various different types of vaginal moisturizers and personal lubricants as one can see by accessing the web site of Shopinprivate.com™ (www.shopinprivate.com/buyinprivate/vagmoisandfe.html). These moisturizers and lubricants are generally water based and sold in the form of lotions, creams, gels and vaginal inserts. These personal products comprise mixtures of various lubricants, moisturizers and preservatives, some of them comprising natural compounds such as vitamin E and aloe vera. Others known lubricants comprise anti-viral agents such as zinc salts (see U.S. Pat. No. 5,785,054). However, none of the genital lubricating compositions existing before the present invention comprise fatty acids or homeopathic dilutions of plant or animal extracts.

The use of natural plant extracts is becoming more and more popular. For instance, CANOLIO INC. (Montreal, QC, Canada) has recently started to sale hemp-based products (coffee, soaps, massage oils, moisturizing body milks, foamy bath oils). Other companies have incorporated hemp by-products in foodstuffs (beer, tortilla chip, salad dressing, pasta, candies, dietary supplements, butter, etc), skin-care products (hand and body lotion, mineral bath salts) and animals products (dog shampoo and animal bedding) (see U.S. Pat. No. 5,827,510; EP 0 856 304; WO 95/31176; Canadian Grocer, March 2000, p 49-50; Oomah and Mazza, Trends in Food Science & Technology 10, 1999, p193-198). Similarly, homeopathic dilutions of plant and animal extracts are been used more and more often since an increasing amount of evidences shows that homeopathic compositions have medicinal properties (Guermonprez M., Matière médicale homeopathique (1989), Editions Boiron, France; Clarke J. -H, A dictionary of practical materia medica (1992), B. Jain Publisher Ltd, New Delhi; Tetau M., Matière médicale homéopathique clinique et associations biothérapeutiques (1979), Maloine Editeur, Paris, France; Reckeweg H-H., Materia Medica (1983), Vol I, Aurelia-Vellag GmbH, Baden-Baden, Germany). However, hemp by-products and medecinal homeopathic compositions have never been incorporated in genital lubricating compositions up to date.

The problems with the genital lubricating compositions to date have been that they are less than optimum for providing an effective relief of vaginal dryness. Furthermore, none of the existing genital lubricating compositions have been helpful in circumventing problems such as loss of libido, frigidity, lack of vaginal lubrication, impotence, and premature ejaculation. Also none of the existing lubricating composition brings polyunsaturated fatty acids and essential fatty acids to the body.

In view of the above, there is a need for genital lubricating compositions comprising fatty acids and/or homeopathic dilutions of plant extracts in order to resolve the above-identified problems.

The present invention fulfils these needs and also other needs which will be apparent to those skilled in the art upon reading the following specification.

### SUMMARY OF THE INVENTION

The present invention relates to a genital lubricant composition, to the uses thereof and to methods for making the same.

According to an aspect of the invention, it is provided a genital lubricant composition comprising: i) at least one ingredient selected from the group consisting of fatty acids and homeopathic dilutions of plant or animal extracts; and ii) a physiologically acceptable carrier.

According to a preferred embodiment, the genital lubricant composition consists essentially of a mixture of fatty acids in combination with a physiologically acceptable carrier.

According to another preferred embodiment, the genital lubricant composition consists essentially of a homeopathic dilution of plant or animal extracts in combination with a physiologically acceptable carrier.

According to a more specific aspect of the invention, it is provided a genital lubricant composition comprising about 0.05% to about 0.5% hemp seed oil and a physiologically acceptable carrier, so that the composition forms a lotion, a cream a gel or a vaginal insert.

The compositions of the invention may be used as a vaginal moisturizer or as a personal lubricant for use prior or during sexual intercourse. They may also be used for bringing polyunsaturated fatty acids and essential fatty acids to the body.

According to another aspect of the invention, it is provided a method for preventing or treating vaginal dryness, comprising the step of applying the genital lubricant composition of the invention inside the vagina.

According to a further aspect of the invention, it is provided a method for preparing a genital lubricant composition comprising the steps of:

a) providing at least one ingredient selected from the group consisting of fatty acids and homeopathic dilutions of plant extracts; and

b) processing this(these) ingredient(s) with a physiologically acceptable carrier so that it forms a lotion, a cream or a gel.

An advantage of the genital lubricating composition of the present invention is that it provides safe and immediate relief of vaginal dryness when used as a vaginal moisturizer. Its ingredients also provide synergetic benefic effects for circumventing problems such as loss of libido, frigidity, lack of vaginal lubrication, impotence, and premature ejaculation when use on a regular basis.

Other objects and advantages of the present invention will be apparent upon reading the following non-restrictive description of several preferred embodiments.

### DETAILED DESCRIPTION OF THE INVENTION

The present invention relates to a genital lubricant composition, and to methods of preparing and using the same.

According to an aspect, the invention provides a genital lubricant composition which comprises i) fatty acids and/or homeopathic dilutions of plant extracts, and ii) a physiologically acceptable carrier.

The genital lubricant composition of the present invention is particularly useful when used as a vaginal moisturizer, or when used as a personal lubricant for use prior or during sexual intercourse. Vaginal moisturizers incorporating the composition of the invention would provide safe and immediate relief of vaginal dryness. Vaginal dryness can occur due to a variety of reasons, including menopause, stress, medication use or even after the use of tampon. Accordingly, in a related aspect of the invention, it is provided a method for preventing and/or treating vaginal dryness by applying the genital lubricant composition of the invention inside the vagina. Similarly, personal genital lubricants including the composition of the invention would be helpful in preventing/diminishing the sexually related problems such as loss of libido, frigidity, lack of vaginal lubrication, impotence, and premature ejaculation when use on a regular basis.

For use, a small quantity (such as a teaspoon or several milliliters) of the composition would be spread across one or more genital surfaces, such as surfaces inside the vagina or the surface of the penis in a manner which causes the lubricant gel to coat and remain in contact with the genital surfaces. It is also conceivable according to the present invention to make lubricated condoms coated with the composition of the present invention since it may helps to increase condoms elasticity and resistance as it will be shown hereinafter in Example 2.

In one preferred embodiment, the genital lubricant composition consists essentially of a mixture of fatty acids (natural or synthetic), in combination with a physiologically acceptable carrier. The fatty acids are preferably essential fatty acids such as linoleic acid (C18:2 or Omega-6) and α-linolenic acid (C18:3 or Omega-3). According to a preferred embodiment, linoleic acid and α-linolenic acid are present in the composition in the ratio of about 1 to 3. The fatty acids may be isolated or purified from plants or from other sources such as from fish. The fatty acids may also be synthesized chemically using well known methods.

Preferably, the fatty acids are provided by incorporating from about 0.01% vol./vol. to about 3% vol./vol. of hemp seed oil to the genital lubricant composition, and more preferably from about 0.05% vol./vol. to about 0.5% vol./vol. Hemp seed oil is preferred since it has been found that it possesses numerous properties which are highly beneficial to the genital lubricant composition of the invention. More particularly, hemp seed oil greatly helps in moisturizing and regenerating the skin and the mucous surfaces. Hemp seed oil has, among vegetable oils, the lowest rate of saturated fatty acids and the highest rate of polyunsaturated fatty acids. Hemp seed oil is further the only known vegetable oil with a ratio of 1 to 3 of linoleic acid and α-linolenic acid, which is the best ratio for human's skin absorption. It also comprises many vitamins (B1, B2, B3, C, D, E) which may be absorbed by the body and it possesses antimicrobial and anti-oxidative properties. Hemp seed oil may be extracted by a first cold pressure of seeds according to the method described by Deferne and Pate (Journal of the International Hemp Association, 1996, 3:1, p 4-7), by organic solvents as described by Kamal and El-Aaser (J. Am. Oil Chemists Soc., 1973, Vol. 50, Abstract #10) or by any other suitable method.

More preferably, the genital lubricant composition further comprises homeopathic dilution(s) of plant extracts. Furthermore, according to another preferred embodiment, the genital lubricant composition consists essentially of a homeopathic dilution of plant extracts in combination with a physiologically acceptable carrier.

Advantageously, the plants and animal(s), from which homeopathic dilutions are prepared, are selected from the group consisting of Caladium seguinum, Sepia officianalis, Lycopodium clavatum, and Onosmodium virginanium, Conium maculatum, Amanita muscaria, Agnus castus and Gelsemium sempervirens are other plants from which extracts may be taken. Homeopathic dilution(s) of these plants and animal(s) are believed to help in preventing/diminishing sexually related problems such as loss of libido, frigidity, lack of vaginal lubrication, impotence, and premature ejaculation (Guermonprez M., Matière médicale homeopathique (1989), Editions Boiron, France; Clarke J. -H, A dictionary of practical materia medica (1992), B. Jain Publisher Ltd, New Delhi; Tetau M., Matière médicale homéopathique clinique et associations biothérapeutiques (1979), Maloine Editeur, Paris, France; Reckeweg H-H., Materia Medica (1983), Vol I, Aurelia-Vellag GmbH, Baden-Baden, Germany). The present inventors also submits that the beneficial activity of the homeopathic dilution(s) is even more important (synergic effects) when used in combination with hemp seed oil. Preferably, the lubricant composition comprises an effective amount of homeopathic dilution(s) so that it will help in improving sexual intercourse when the composition is applied to genital areas. Of course, the exact amount may vary according to different factors such as the nature of the extracts and the condition to be treated. According to a preferred embodiment, the lubricant composition comprises about 1% vol./vol. of homeopathic dilution(s) of plant extracts.

Preferably, the composition should forms a lotion, a cream or a gel and the physiologically acceptable carrier should have: i) water; ii) a thickening agent; and iii) a lubricating agent. The composition may also be in the form of a vaginal insert. In order to be physiologically acceptable, the selected carrier should be formulated so that it does not cause any significant adverse effects (such as irritation, tenderness, swelling, redness, or skin discoloration), and it does not pose a significant risk as a carcinogen or teratogen.

In contrast to non-physiological lubricants such as motor oil, physiologically acceptable lubricating agents should be either gradually broken down into innocuous substances in the body if they are absorbed by tissue to a significant degree through the skin or mucous membranes, or they should be of a nature that allows them to be secreted by the vagina and washed cleanly from the skin, so that they will not foul and clog the pores in membranes or dermal layers. Several lubricating agents which are used in commercially available sexual lubricants satisfy these criteria, including glycerin (also called glycerine, glycerol, 1,2,3-propanetriol, and trihydroxypropane) and certain types of polyethylene glycol (PEG), such as PEG 200 or PEG 400 (the numbers indicate different molecular weight averages). Various other polymers (such as polypropylene glycol, polyisobutene, and polyoxyethylene) and behenic acid and behenyl alcohol are also used as lubricants in cosmetics and other formulations that contact the skin. In addition, some sugar-alcohols such as sorbitol, and some silicon compounds such as polydimethylsiloxane, are also used as skin-contacting lubricating agents.

Because glycerin, propylene glycol, polyethylene glycol, and polypropylene glycol have long been used in sexual lubricants and other skin-contacting formulations with no adverse effects, they are preferred for use as lubricating agents in the composition of this invention. The suitability of any other candidate lubricating agent can be determined through routine experimentation in humans to ensure that it will not cause irritation or other adverse effects, and in in vitro cell culture and in in vivo lab animal tests.

A suitable thickening agent which is widely used in genital lubricants comprises chemically treated derivatives of cellulose (such as hydroxyethyl- or hydroxymethyl-cellulose). Other thickening agents which have been used in skin-contacting compounds, and which offer candidate agents for potential use in genital lubricant compositions, include acacia, agar, alginate, carrageenan, gum tragacanth, xanthan gum, collagen, carboxypolymethylene, glyceryl monostearate, polyvinylpyrrolidone, and polyacrylamide.

Other components, including preservatives (such as DMDM hydantoin, chlorhexidine gluconate), anti-crystallization agents (such as glucono-delta-lactate), fragrances, sweeteners, odorants, coloring agents, alkaline or acidic or buffering agents to maintain the proper pH (such as EDTA), and soothing, anti-swelling agents (such as lanolin, aloe vera extract, or hydrocortisone), antiviral agents (such as zinc salts; see U.S. Pat. No. 5,785,054), hormones (such as estrogen) or spermicide (such as nonoxynol-9) can be added to the lubricant composition of the invention described herein. However, at the concentrations used, any such additive should not seriously impede the desired activity of the final composition and should not irritate or have other adverse effects on the genitals.

The complete mixture must be physiologically safe and acceptable when used repeatedly over a period of months or years, and it must not irritate mucous membranes or other genital surfaces even when rubbed in vigorously (during intercourse for instance). The composition should also be free of anti-coagulants (particularly heparin or dextran sulfate) or other components which could pose a risk of adverse effects in a significant portion of the population.

### EXAMPLES

The following examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

### Example 1

### Preparation of a Personal Genital Lubricant

A personal genital lubricant in the form of a liquid gel comprising the followings ingredients was prepared:

| Ingredients: | Final Concentration (Vol/Vol): | |
|---|---|---|
| 1-Water | about | 90% |
| 2-Glycerin | about | 7% |
| 3-Hydroxyethyl cellulose | about | 1% |
| 4-DMDM hydantoin | about | 0.25% |
| 5-Tetrasodium EDTA | about | 0.15% |
| 6-Methyl paraben | about | 0.1% |
| 7-Citric acid | about | 0.05% |
| 8-Hemp seed oil | about | 0.05% |
| 9-Aqueous Homeopathic dilution | about | 1% |
| of plant or animal extracts* | | |

*(mixture of Caladium seguinum, Sepia officianalis, Lycopodium clavatum, and Onosmodium virginanium extracts)

The aqueous homeopathic dilution to be used in the liquid gel was prepared by macerating the plant and animal extracts in 98% alcohol. The resulting mixture was diluted 100×with water and agitated. The dilution and agitation step was repeated five times to obtain a five centesimal dilution of the original alcoholic mixture. This diluted aqueous solution was then incorporated in the personal genital lubricant.

The above mentioned liquid gel was prepared using a conventional method known in the art. Briefly, ingredients No 5, 6 and 4 were added on at the time, under agitation, to a tank with water. Thereafter, ingredients No 7, 2, 3, 8 and 9 were also added, one at the time, under constant agitation. The resulting liquid gel was petroleum free, water soluble, irritant-free, non-sticky, non-staining, unscented and without after-taste. It was also easy to apply and it provided an increased fluidity during intercourse.

### Example 2

### Increased Condoms Resistance

The composition of the invention was tested (burst resistance test) to confirm that it was compatible with latex condoms.

Briefly, ultra-thin lubricated latex condoms and around regular non-lubricated condoms were coated with a large amount of the liquid gel prepared as described in Example 1. After one hour at 37° C., air was then pumped into the condoms until burst for evaluating and comparing condoms resistance to burst. Unexpectedly, the liquid gel of the invention was seen to increase condoms resistance and elasticity, as shown in Table 1 hereinafter.

TABLE 1
Burst resistance test

| | Burst resistance of condoms | |
|---|---|---|
| | ultra-thin lubricated | regular non-lubricated |
| Without any gel | ++ | ++ |
| With the liquid gel | +++ | ++ |
| of the invention | | |

These results show that the liquid gel of the invention was suitable with condoms. Interestingly, these results also shows that it could be useful for increasing the resistance and elasticity of the condoms, a very interesting advantage of the present invention

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

What is claimed is:

1. A genital lubricant composition comprising:
i) at least one ingredient selected from the group consisting of fatty acids and homeopathic dilutions of plant or animal extracts; and
ii) a physiologically acceptable carrier.

2. The genital lubricant composition of claim 1, wherein it consists essentially of a mixture of fatty acids in combination with a physiologically acceptable carrier.

3. The genital lubricant composition of claim 2, wherein said fatty acids are isolated or purified from plants.

4. The genital lubricant composition of claim 2, wherein said fatty acids are essential fatty acids.

5. The genital lubricant composition of claim 2, wherein it comprises hemp seed oil.

6. The genital lubricant composition of claim 5, comprising from about 0.01% vol./vol. to about 3% vol./vol. hemp seed oil.

7. The genital lubricant composition of claim 6, comprising from about 0.05% vol./vol. to about 0.5% vol./vol. hemp seed oil.

8. The genital lubricant composition of claim 2, wherein said fatty acids comprise linoleic (C18:2) and linolenic (C18:3) fatty acids.

9. The genital lubricant composition of claim 8, wherein said linoleic and linolenic fatty acids are present in the ratio of about 1 to 3.

10. The genital lubricant composition of claim 2, further comprising a homeopathic dilution of plant extracts.

11. The genital lubricant composition of claim 2, wherein it forms a lotion, a cream, a gel or a vaginal insert.

12. The genital lubricant composition of claim 2, wherein said physiologically acceptable carrier comprises water, a thickening agent and a lubricating agent.

13. The genital lubricant composition of claim 2, for use as a vaginal moisturizer, or as a personal lubricant for use prior or during sexual intercourse.

14. The lubricant composition of claim 1, wherein it consists essentially of a homeopathic dilution of plant or animal extracts in combination with a physiologically acceptable carrier.

15. The lubricant composition of claim 14, wherein said plant(s) or animal(s) are selected from the group consisting of Caladium seguinum, Sepia officianalis, Lycopodium clavatum, and Onosmodium virginanium.

16. The lubricant composition of claim 14, wherein it comprises an effective amount of said homeopathic dilution so that, when applied to the genitalia, it reduces sexually related problems associated with sexual intercourse.

17. The lubricant composition of claim 14, comprising about 1% vol./vol. of said homeopathic dilution.

18. The genital lubricant composition of claim 14, further comprising from about 0.05% vol./vol. to about 0.5% vol./vol. hemp seed oil.

19. The lubricant composition of claim 14, wherein it forms a lotion, a cream, a gel or a vaginal insert.

20. A genital lubricant composition comprising about 0.05% to about 0.5% hemp seed oil in combination with a physiologically acceptable carrier so that it forms a lotion, a cream, a gel or a vaginal insert.

21. A method for preventing or treating vaginal dryness, comprising the step of applying the genital lubricant composition of claim 1 inside the vagina.
